(19) United States
(12) Patent Application Publication
(10) Pub. No.: US 2005/0163685 A1
(43) Pub. Date: Jul. 28, 2005

(54) Pre-sterilisation chamber for a processing enclosure

(76) Inventors: Donald Kerr Bissell, Surrey (GB); James Lindsay Drinkwater, Buckinghamshire (GB)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/512,629

(22) PCT Filed: Sep. 23, 2003
(86) PCT No.: PCT/GB03/04087

(30) Foreign Application Priority Data
Sep. 24, 2002 (GB) 0222154.7

(51) Int. Cl. A61L 2/20

(52) U.S. Cl. 422/292; 422/300

(57) ABSTRACT

The disclosure relates to an ante-chamber for pre-sterilising components/materials to be supplied to a processing enclosure (e.g., an isolator enclosure, room, cabinet or the like) the ante-chamber having a closable entry for receipt of components/materials and a closable exit for supply of materials/components to the isolator enclosure, valve control and supply and return conduits for sterilant vapour for sterilizing the chamber and its contents and valve controlled supply and purge gas conduits for purging the chamber of sterilant at the end of the sterilizing operation. The supply and return conduits having filters to filter out particles from the air being delivered to the chamber and recovered from the chamber respectively. The valves for controlling the supply and return conduits are disposed between the filters and enclosure, the arrangement being such that the supply and return conduits for purge gas may be arranged to receive sterilant vapour periodically to sterilize the conduits.

This invention relates to pre-sterilisation ante-chamber for processing enclosures such as isolator enclosures, rooms, cabinets or the like in which processing operations are conducted under sterile conditions.

The object of the invention is to provide rapid surface gaseous sterilisations of components and material within a chamber so that the surfaces of the said components and materials may be rendered sterile. The components and materials may then be transferred from the chamber into a sterile processing area without the risk of causing contamination within the processing area.

Typically when small numbers of aseptic drug preparations are required they are dispensed either in a hospital pharmacy or a pharmacy facility that serves a hospital. Normally the components and material required for the dispensing are placed in an isolator for aseptic processing. The surfaces inside the isolator are bio-decontaminated, generally by using a gaseous process; the drugs are then dispensed and removed from the isolator. The problem with this technique is that because the sterilising cycle is long it is necessary to place sufficient components and material a inside the isolator for one whole day's work. The workload must therefore be planned the previous day making it difficult to respond to emergencies and changes in requirements, making the process very inflexible. Large banks of pre-sterilised material are often therefore used to improve the flexibility of response, but this approach is space consuming and expensive, with long recovery times in the event of loss of sterility of the bank isolator.

The main reason that the decontamination process is long is the absorption of the sterilizing gas into the surface of the components and material forming the load and also the surfaces of the chamber including the HEPA filters used to provide a stream of sterile air to the chamber. If the size of the load can be reduced and HEPA filters not exposed to the gas during routine process transfer much shorter cycles times would result, thus giving the required flexibility to bio-decontaminate components and material on demand. Removing the HEPA filters from the space that is bio-decontaminated with the components and material creates a further problem, in that all surfaces that come into contact with the air entering or leaving the chamber must be sterile, or these surfaces will be a source bio-contamination that may enter the chamber and hence contaminate the product.

The invention provides an ante-chamber for pre-sterilising components/materials to be supplied to a processing enclosure (e.g. an isolator enclosure, room, cabinet or the like) the ante-chamber having a closable entry for receipt of components/materials and a closable exit for supply of materials/components to the isolator enclosure, valve control and supply and return conduits for sterilant vapour for sterilising the chamber and its contents and valve controlled supply and purge gas conduits for purging the chamber of sterilant at the end of the sterilising operation, the supply and return conduits having filters to filter out particles from the air being delivered to the chamber and recovered from the chamber respectively and the valves for controlling the supply and return conduits being disposed between the filters and enclosure, the arrangement being such that the supply and return conduits for purge gas may be arranged to receive sterilant vapour periodically to sterilise the conduits.

A greater degree of flexibility is achieved by using a relatively small chamber on the side of the dispensing isolator, and devising a rapid surface sterilization process for the product and components inside the chamber. By reducing the sterilization time to less than 20 minutes it becomes possible to generate a flow of material through the small chamber into the working isolator and thus give a greater degree of flexibility to the operations. To achieve such a short cycle time it is essential to arrange that surface decontamination is achieved in about 6 minutes and that aeration, the removal of the sterilant gas is achieved in 14 minutes.

Surface sterilization will only be achieved in such a short period if the gas injection rate is high and the gas distribution within the chamber is carefully managed to achieve even gas distribution at even gas temperatures.

To achieve rapid aeration, high purge air rates are required but of equal importance is to ensure that there are no absorbent surfaces, such as HEPA filters, in contact with the gas, during the load sterilisation.

Preferably a valve controlled supply of sterilant is provided for the purge gas supply conduit for supplying sterilant vapour through the conduit and to the return conduit via the ante-chamber to sterilise the purge gas supply and return conduit.

In the latter case the valve for controlling the supply of sterilant to the purge gas supply conduit may be located upstream of the filter in the conduit.

In either of the latter arrangements the return conduit for purge gas from the chamber may have a catalyst downstream of the filter for converting the sterilant into products which may be discharged to atmosphere.

More specifically a further filter may be located in the return conduit downstream of the catalyst to remove any particle in the purge gas received from the catalyst.

In any of the above arrangements the sterilant gas supply conduit chamber may have a fan for delivering air to the ante-chamber via the filter and valve to purge sterilant gas from the chamber.

Also in any of the above arrangements, the return conduit for purge gas may have a fan for extracting purge gas from the chamber disposed downstream of the valve control and filter.

Furthermore the supply and return conduits for purge gas both contain a pair of filters and a catalyst for converting sterilant to harmless products disposed between the filters and the valves are arranged to open both return and supply conduits to atmosphere for delivery of sterilant gas from the ante-chamber to sterilise the supply and return conduits.

The following is a description of some specific embodiments of the invention, reference being made to the accompanying drawing in which:

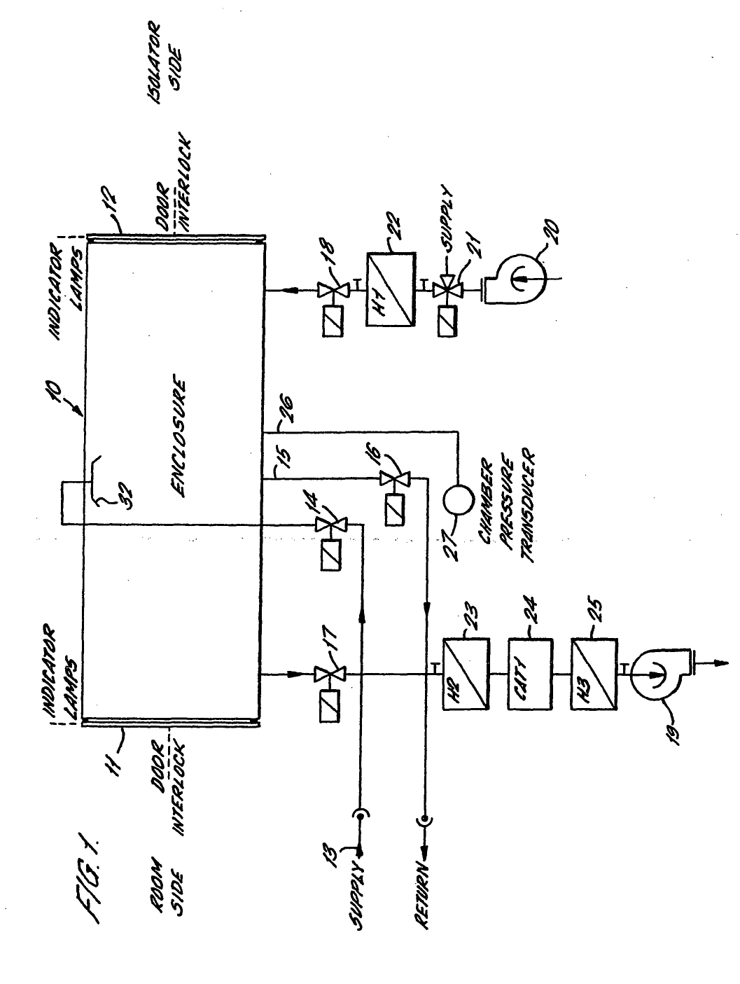

FIG. 1 is a diagrammatic illustration of an ante-chamber for pre-sterilising components/material before entry to a sterile processing enclosure;

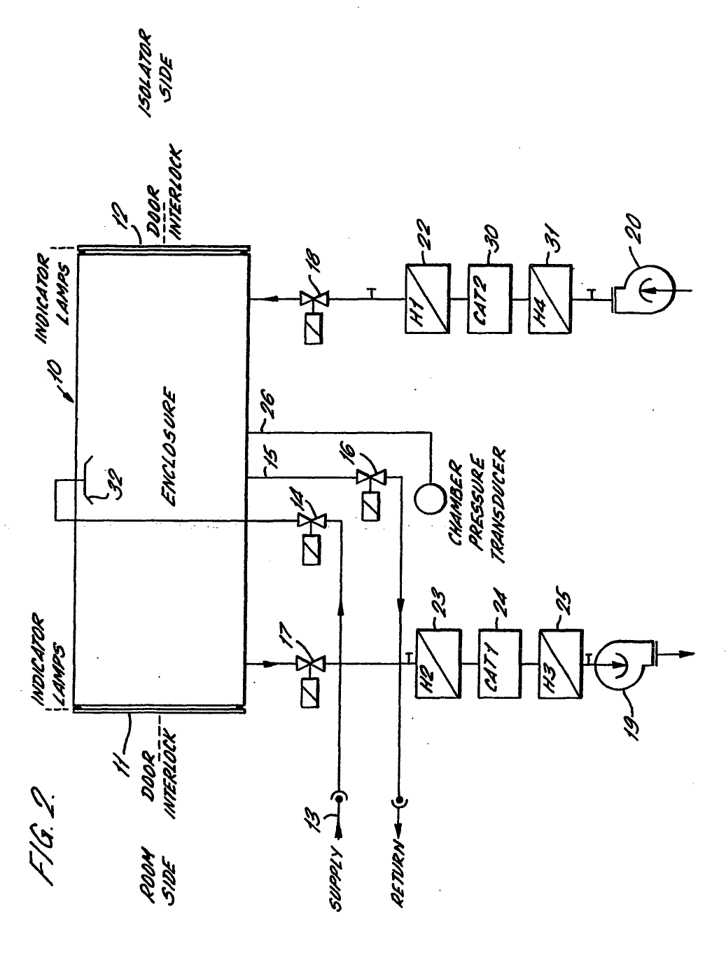

FIG. 2 is a diagrammatic illustration of a second ante-chamber for pre-sterilising material; and

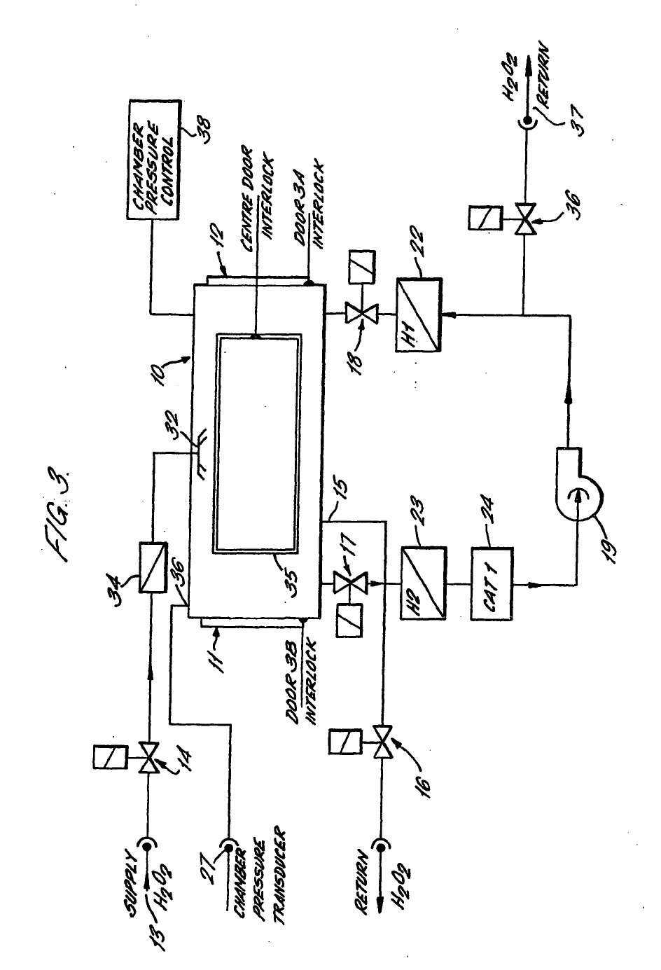

FIG. 3 is a diagrammatic illustration of the ante-chamber of FIG. 1 embodied in a closed loop system.

The components and material, known as the load, to be bio-decontaminated are placed inside a chamber 10 through a first chamber door 11. At the other end of the chamber 10 is a second door 12 connected to a dispensing isolator (not shown) or processing enclosure. It is preferred that the first and second doors are provided with interlocks such that only one door may be opened at a time and also so that a door may only be opened when the atmosphere inside the chamber 10 is safe. Indication lamps are provided adjacent each door to indicate the state of opening/closure of the doors.

Once the load is placed inside the chamber and the first and second doors are closed and sealed, sterilizing gas is introduced into the chamber via a port 13 which connected through a valve 14 to the chamber. At this time the valve 14 must be opened to allow the gas to enter the chamber. The sterilizing gas is removed from the chamber through a port 15 controlled by a valve 16. The most commonly used sterilizing gas is hydrogen peroxide, and generally the commercially available hydrogen peroxide gas generators operate as a close of loop system with the gas returning to the generator.

During the circulation of the sterilizing gas further valves 17 and 18 which are connected to the chamber remain closed. Once the gaseous sterilization phase has been completed and it is required to remove the gas from the chamber the valves 17 and 18 are opened and fans 19 and 20 are switched on. At this point a 3-way valve 21 is set to deliver air from fan 20 to the valve 18.

The fan 20 takes air from the surrounding environment passing it through the 3-way valve 21 and a HEPA filter 22 and valve 18 into the chamber. This fresh air will reduce the gas concentration in the chamber by dilution. An equal quantity of air must be removed from the chamber through the valve 17, HEPA filter 23, a catalytic filter 24 and a further HEPA filter 25, by fan 19. It is important that the air fed into the chamber by fan 20 is filtered through the HEPA filter 22 to ensure that the chamber and the load inside the chamber remains sterile after gassing. Also on the exhaust side the air removed from the chamber must pass firstly through a HEPA filter 23 to stop any particles escaping back into the chamber and rendering it non-sterile. The catalytic filter 24 is used to render the exhaust gas safe before it is passed through the further HEPA filter 25 to remove any dust particles and then back into the surrounding environment.

A further connection 26 to the chamber is required for a pressure transducer 27 to monitor the pressure inside the chamber. A small HEPA filter (not shown) in the connection 26 avoids any contamination of the chamber from the connection. The pressure as measured by the transducer 27 is used to control fans 19 and 20 to achieve the required pressure in the chamber. Fans 19 and 20 are adjusted to achieve an airflow through the chamber at sufficiently high flow rate to remove the sterilizing gas in about 15 minutes. Experiment has shown that this will require an air change rate of about 2000 per hour.

Because of the need to ensure that the hydrogen peroxide gas does not come into contact with the HEPA filters 22 and 23 there is a space in the conduit between the filter 23 and valve 17, and also a further space between the filter 22 and valve 18 which is not sterilized. This space forms part of the air path during the aeration of the cycle. Any contamination in these spaces may therefore be transferred to the chamber and hence may contaminate the load within the chamber.

Two possible techniques are available to ensure that these spaces are bio-decontaminated and hence do not pose a risk to the load. The first will now be described by reference to FIG. 1. The hydrogen peroxide gas supply is connected to the 3-way valve 21 such that the gas flows into the valve and thence to the chamber via the HEPA filter 22 and the valve 18, which must be open. The valves 14 and 16 are closed and the valve 17 opened to allow the gas to pass out through the HEPA filter 23, the carbon filter/catalyst 24 which renders the gas safe, through the HEPA filter 25 and finally exhausting through the fan 20. The passage of gas from the 3 way valve 21 through the chamber 1 and out through the fan 19 is allowed to continue for sufficient length of time to ensure decontamination of all of the components in this flow path.

At the end of the period the system is put back into aeration, as before, to remove the hydrogen peroxide vapour. Because this air path is protected by HEPA filtration it will require bio-decontamination at infrequent intervals, probably once every two weeks, depending on the usage of the chamber.

The second technique will now be described with reference to FIG. 2.

With this technique hydrogen peroxide gas is supplied from the generator through valve 14 into the chamber. The valve 16 remains closed and valves 17 and 18 are opened, allowing the gas to flow from the chamber through two pathways. The gas leaves the chamber either through valve 17 or valve 18. The gas leaving through valve. 18 passes through a HEPA filter 22 a filter/catalyst 30 where the gas is rendered safe. The exhaust gas then passes through further filter 31 HEPA 4 and finally exits the system through fan 20. The other stream of gas leaving through valve 17 passes through HEPA filter 23, filter/catalyst 24 and the filter HEPA 25. By passing through the filter/catalyst 24 the gas is rendered safe before returning to the room through the fan 19. This gas flow is maintained for a sufficient period of time to ensure that the whole of the flow path is bio-decontaminated. Once sufficient time has elapsed then the system may be returned to aeration mode to remove the hydrogen peroxide gas.

Because gas distribution within the chamber and around the load is very important it is sensible to use some device to give the gas some kinetic energy when entering the chamber. This may be achieved by using a rotating nozzle 32, which not only ensures that the gas enters the chamber at high velocity but also changes the direction of the jet. This also avoids the problem associated with causing hot spots as a static gas jet impinges on a small area of a surface.

Alternatively the rotating nozzle 32 may be replaced with either a fixed nozzle or a number of fixed nozzles that ensure good gas distribution.

A loading system will be required to place the load into and remove it from the chamber. A suitable system would be a trolley/rack that can be partially withdrawn from the chamber through the outer door to assist with loading the chamber. After sterilisation the trolley/rack system can then withdrawn into the processing enclosure through the inner door where it may be unloaded.

The chamber and all of the associated components should form one integrated self-contained unit that may be constructed as a mobile device capable of being moved to interface with various process enclosures.

Reference is now made to FIG. 3 which shows a closed loop system. The numbering system of FIGS. 1 and 2 is utilised in FIG. 3, like parts being allotted the same reference numerals. The closed loop system avoids the need to exhaust air during the aeration phase. This has the advantage that should there be a failure in the catalytic destruction of the active gas then no toxic gas would be released into the room or environment. It also simplifies leak testing of the system as the number of potential leak paths is reduced.

The chamber of FIG. 3 has up to three doors. One 11, 12 on each end as before to allow connection to two isolators and a third 35 in the centre through which the components to be sterilised are loaded. Each of these doors is fitted with a sensor to indicate when they are open or closed and a mechanism to ensure that only one is open at any time.

The gassing (sterilisation) process is the same as in FIGS. 1 and 2. Biodecontamination of the aeration pathway that is not sterilised during normal gassing is achieved by closing valves 7 and 8 and opening valves 17 and 35. The gas supply is then connected to valve 5 and the return to 37. This causes the sterilising gas to pass from the chamber through valve 5 and HEPA filter 13 thus exposing those surfaces not exposed to gas during the normal cycles.

Following the gassing cycle valves 8 and 9 are opened and the fan 11 is started. This generates a large air flow through the filter 23 and the catalytic destructor 24 that renders the active gas safe. After passing through the fan the air passes through a second HEPA filter 13 to remove any particulate contamination that may have arisen from the catalytic destructor or the fan. Because of the very high air flow (approximately 2000 to 3000 air changes per hour) through the catalytic destructor 24 the gas concentration in the chamber 10 is rapidly reduced to a safe level.

1-8. (canceled)

9. An ante-chamber for pre-sterilising components/materials to be supplied to a processing enclosure, the ante-chamber comprising a closable entry for receipt of components/materials and a closable exit for supply of materials/components to the isolator enclosure, valve control and supply and return conduits for sterilant vapour for sterilising the chamber and its contents and valve controlled supply and purge gas conduits for purging the chamber of sterilant at the end of the sterilising operation, the supply and return conduits having filters to filter out particles from the air being delivered to the chamber and recovered from the chamber respectively and the valves for controlling the supply and return conduits being disposed between the filters and enclosure, the arrangement being such that the supply and return conduits for purge gas may be arranged to receive sterilant vapour periodically to sterilise the conduits.

10. An ante-chamber as claimed in claim 9, wherein a valve controlled supply of sterilant is provided for the purge gas supply conduit for supplying sterilant vapour through the conduit and to the return conduit via the ante-chamber to sterilise the purge gas supply and return conduit.

11. An ante-chamber as claimed in claim 10, wherein the valve for controlling the supply of sterilant to the purge gas supply conduit is located upstream of the filter in the conduit.

12. An ante-chamber as claimed in claim 10, wherein the return conduit for purge gas from the chamber has a catalyst downstream of the filter for converting the sterilant into products which may be discharged to atmosphere.

13. An ante-chamber as claimed in claim 12, wherein a further filter is located in the return conduit downstream of the catalyst to remove any particle in the purge gas received from the catalyst.

14. An ante-chamber as claimed in claim 9, wherein the sterilant gas supply conduit chamber has a fan for delivering air to the ante-chamber via the filter and valve to purge sterilant gas from the chamber.

15. An ante-chamber as claimed in claim 9, wherein the return conduit for purge gas has a fan for extracting purge gas from the chamber disposed downstream of the valve control and filter.

16. An ante-chamber as claimed in claim 9, wherein the supply and return conduits for purge gas both contain a pair of filters and a catalyst for converting sterilant to harmless products disposed between the filters and the valves are arranged to open both return and supply conduits to atmosphere for delivery of sterilant gas from the ante-chamber to sterilise the supply and return conduits.
